(19) United States
(12) Patent Application Publication
(10) Pub. No.: US 2022/0280795 A1
(43) Pub. Date: Sep. 8, 2022

(54) Wireless Closed Loop Deep Brain Stimulation Method and System

(71) Applicant: Sensoria Therapeutics, Inc., Irvine, CA (US)

(72) Inventor: Ross Tsukashima, Irvine, CA (US)

(73) Assignee: Sensoria Therapeutics, Inc., Irvine, CA (US)

(21) Appl. No.: 17/686,311

(22) Filed: Mar. 3, 2022

Related U.S. Application Data
(60) Provisional application No. 63/156,279, filed on Mar. 3, 2021.
(60) Provisional application No. 63/166,284, filed on Mar. 26, 2021.

(51) Int. Cl. A61N 1/36; A61B 5/11; A61B 5/00

(52) U.S. Cl. CPC A61N 1/36067; A61B 5/1101; A61B 5/6824; A61B 5/746; A61B 5/0004; A61B 5/4082

(57) ABSTRACT

A wireless deep brain stimulation method and device.

This application claims priority to U.S. Provisional Application 63/156,279 filed Mar. 3, 2021 and U.S. Provisional Application 63/166,284 filed Mar. 26, 2021.

### FIELD OF THE INVENTIONS

The inventions described below relate to devices and methods that provide treatment of movement disorders.

### BACKGROUND OF THE INVENTIONS

Deep brain stimulation (DBS) technology has shown promise for treatment of movement and effective disorders such as Parkinson's disease, epilepsy, essential tremor and dystonia. Typical protocols use multiple sensor probes and stimulation probes attached to wiring that is connected to an implantable pulse generator. Limitations on the number of connections to the pulse generator limits closed loop sensing capability on wired systems. There is a need for a deep brain stimulating system and treatments for movement disorders that includes both the sensing of native brain signals and stimulation of the brain based on sensed reading from conditions within the brain or from motion sensors placed on patient body parts.

### SUMMARY

The devices and methods described below provide for improved treatment of movement disorders using senor probes and stimulation probes operated by an external control system. The control system includes a transmitter and a receiver. The control system is operable outside the patient's body to receive signals from the sensor probes indicative of a movement disorder. The control system receives the data and analyzes whether the signals are within a predetermined threshold or range. If the control system determines that the received signals are outside of a predetermined threshold, it delivers control signal to the stimulation probe inside the brain to transmit a stimulation pulse to structures within the brain to effect therapeutic changes in the brain.

The devices and method can also include a motion sensor mounted on a body part subject to aberrant motion characteristic of a motion disorder. The motion sensor detects tremors or other symptoms associated with a movement disorder. Motion signals obtained from motion sensors to the control system to calculate and deliver electrical stimulation. The control system is operable to generate and transmit control signals to the probes, based on the motion signals in order to cause the probes to transmit stimulation pulses to structure within the brain to effect therapeutic changes.

A method of treating movement disorders with stimulation of a patient's brain is also disclosed. The method is performed by inserting a plurality of stimulation probes with a patient's brain. Motion sensors are also placed on a body part of a patient that is subject to aberrant movement associated with movement disorders. The motion sensors detect a sensed motion signals and generates signals corresponding to the sensed movement of the body part. The sensed motion signal is sent to a control system that detects patterns that result from the sensed signals. The control system sends delivery signals to the stimulation probes to cause the stimulation probe to apply stimulation to a portion of the patient's brain associated with the movement disorder.

### BRIEF DESCRIPTION OF THE DRAWINGS

FIG. 1 illustrates a first wireless closed loop deep brain-stimulating or movement disorder stimulation system.

FIG. 2 illustrates a second movement disorder stimulation system.

### DETAILED DESCRIPTION OF THE INVENTIONS

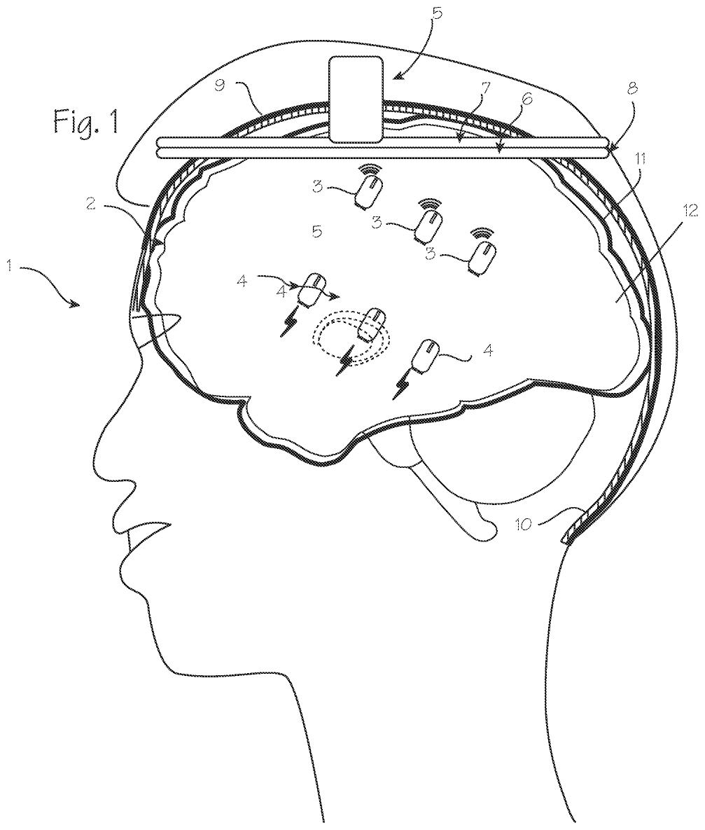

FIG. 1 illustrates a first wireless closed loop deep brain-stimulating or movement disorder stimulation system. A patient 1 with a condition requiring deep brain stimulation (DBS) of the brain 2 is illustrated. FIG. 1 shows the placement of a plurality of sensor probes 3 and stimulation probes 4 disposed within the brain of the patient. An external control system 5 is operable to power, communicate with and control the sensor and stimulation probes within the patient's brain. The external control system includes a transmitter 6 and a receiver 7 disposed outside of the patient's body to receive and send control signals to the stimulation probes in order to stimulate the brain. The control system can be worn by the patient like a hat or a head band 8 on a head. The control system may also be disposed elsewhere near the patient depending on the power and sensitivity of the transmitter and receiver.

The sensor probes and stimulation probes can be inserted entirely within the brain at various positions. FIG. 1 illustrates the scalp 9, which surrounds the brain as an outer layer. The skull 10 is beneath the scalp, the dura 11 is beneath the skull and the cerebral cortex 12 is beneath the dura. Scalp probes can be positioned superficial to the scalp. Subdural probes can be positioned under the dura layer, between the dura and the cortex. Intracortical probes can be positioned beneath the dura on the cortex.

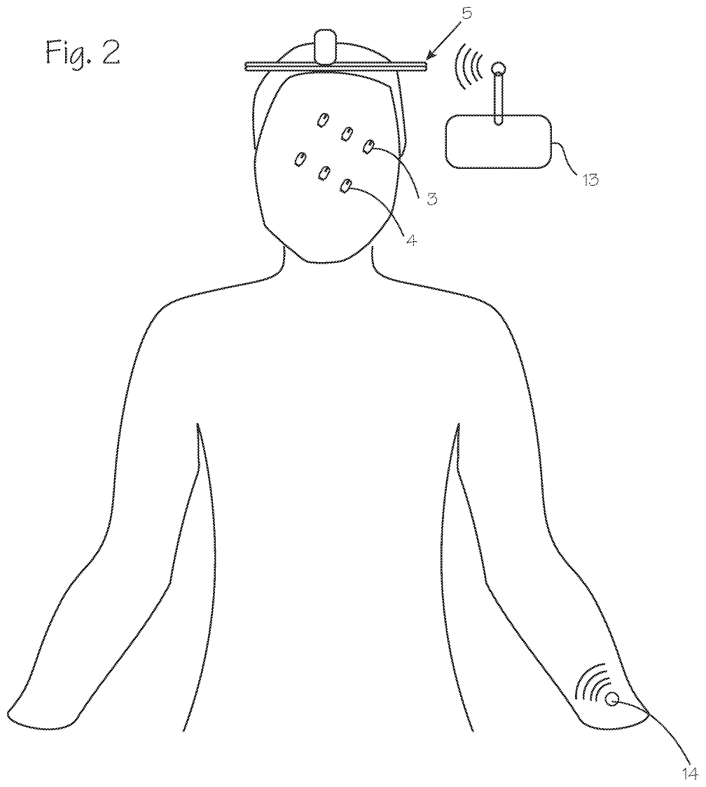

The probes can include sensing electrodes and electronics operable to detect native brain signals, and generate electronic signals corresponding to native biological brain signals and transmit those signals to the control system. The control system 5 is operable to receive electronic signals corresponding to native biological brain signals from the probes and interpret whether the native biological brain signals are within a predetermined band of native biological brain signals, or above or below a predetermined threshold for the native biological brain signals. The control system is also operable to generate and transmit control signals to the probes, to cause the probes to transmit stimulation pulses to structures within the brain to effect therapeutic changes in native biological brain signals (which in turn are indicative of brain activity related to a disorder such as Parkinson's Disease, epilepsy, essential tremor and dystonia). The control system is depicted on the head of the patient, and this location provides for convenient placement and lower power requirements vis-à-vis more remote locations, the control system may be disposed elsewhere on the body of the patient (on the wrist, co-located with the accelerometer shown in FIG. 2, on a belt in a pager-like form factor, implanted in the patient, or disposed remotely from the patient but within range of the communications protocol and limits of the wireless power transmission.

The sensor probes are adapted to sense electrical activity from a patient's brain. The stimulation probes are adapted to deliver electrical stimulation to the patient's brain. Alternatively, a single probe can be used for both sensing of electrical activity from a patient's brain as well as delivery of electrical stimulation to the brain. The single probe performs sensing and stimulation in a duty cycle where a percentage of the time it senses and a different percentage of the time it stimulates. The single probe reports the presence of a specific native biological brain signal or molecular event within the brain by communicating a measurable signal. The measurable signal may be a light-based readout. The single probe separately delivers electronic stimulation to the patient's brain. The control system has a unique address for identification and is externally powered and enables directional communication and control of the sensor and stimulation probes. The probes are spatially dispersed within the brain and operate via spontaneous formations of networks where the data is transmitted wirelessly. The networks are bi-directional, both collecting data from distributed probes as well as enabling control of probe activity. Patients with neurological disorders may exhibit abnormal EEG, ECoG, AP's or LFP signals indicative of their disease. These abnormal signals can serve as biomarkers to be monitored to determine treatment of the patient.

The native biological brain signals correspond to motor deficiencies, which may include Parkinson's Disease, epilepsy, essential tremors and/or dystonia. The native biological brain signals may also be signals such as abnormal hyperactivity in Broadmann's area 25 that correspond to mood disorders such as depression. Therapy for neurological disorders can be used for motor and ocular motor control diseases, but can be applied by monitoring and applying stimulation to areas of the dorsolateral prefrontal and lateral orbitofrontal cortex for associative diseases involved in cognition or memory, or the monitoring and stimulation of the limbic and paralimbic cortex, hippocampus and amygdala for the treatment of limbic ailments such OCD or used for treatment of pain management, stroke rehabilitation and cognition impairment. Monitoring and stimulation can be in different locations of the brain.

Sensor probes 3 inserted within the brain 2 detect and/or record signals linked to symptoms exhibited within the brain. The sensor probes detect EEG, ECoG, AP, LFP or other sensed bio-signals. The sensor probes transmit the electronic signal corresponding to the sensed bio-signal to the receiver of the control system. The control system processes the electronic signal and is operable to transmit control signals to the stimulation probes to cause the stimulation probes to apply stimulation in response to variations in the sensed signals. The sensor probes are operable to sense signals from the thalamus, STN, cortex or other associated structures of the brain, which are indicative of the conditions treated (signals indicative of reduced or increased unwanted motor activity in the patient, for example). Adjustment in the stimulation provided by the stimulation probes can also be made through operator input to the control system, in response to sensed signals from the sensor probes, for example in response to data provided by the control system through an output such as a display screen or audio speakers. Adjustment in the stimulation provided by the stimulation probes may also be made by the control system, without immediate operator input, if the control system is programmed to determine stimulation levels or patterns appropriate to apply or adjust in response to sensed signals from the sense probes. This real-time optimization would allow neurons the chance to rest and thus reduce overall deterioration over time.

FIG. 2 illustrates a second movement disorder stimulation system. This system is operably connected to additional network 13 connected to probes located outside the brain 2 and may include an external motion sensor 14 mounted on a body part subject to aberrant motion characteristic of a motion disorder. The external motion sensor could be an accelerometer or other sensor, mounted on the wrist of the patient 1. The system includes a stimulation probe configured for insertion into the patient's brain at a location subject to stimulation affecting symptoms of the movement disorder. The stimulation probe is operable to apply stimulation to a portion of the patient's brain associated with the movement disorder. The system asl includes a motion sensor configured for placement on a body part of the patient where the body part subject to aberrant movement associated with (characteristic of) a movement disorder. The motion sensor is operable to sense movement of the body part it is placed on and to generate signals corresponding to sensed movement of the body part. The signals corresponding to sensed movement of said body part is transmitted to the control system and the control system is operable to receive the signals. The control system controls the stimulation probe to cause the stimulation probe to apply stimulation to a portion of the patient's brain associated with the movement disorder. The control system is programmed to (1) receive signals corresponding to sensed movement of said body part and determine if the signals corresponding to sensed movement of said body part are indicative of aberrant movement associated with (characteristic of) the movement disorder. The control system is also programmed to control the stimulation probe to cause the stimulation probe to apply stimulation to a portion of the patient's brain associated with the movement disorder. The control system is also programmed to continue to receive signals corresponding to sensed movement of the body part and conditionally (4) determine that the signals corresponding to sensed movement of the body part are indicative of aberrant movement associated with (characteristic of) the movement disorder. The control system can further cause the stimulation probe to cause the stimulation probe apply stimulation or (5) determine that the signals corresponding to sensed movement of said body part are NOT indicative of aberrant movement associated with (characteristic of) the movement disorder, and cease causing the stimulation probe to cause the stimulation probe apply stimulation. The control system is programmed to determine if the sensed motion exceeds a predetermined threshold of tremor characteristic of tremors due to various conditions such as essential tremor, Parkinson's disease epileptic seizures and cause the stimulation probe to apply stimulation to the brain until the sense motion falls below the predetermined threshold.

The system can also include a sensor probe operable to detect electrical activity of the brain indicative of an epileptic seizure and generate signals corresponding to electrical activity of the brain. These signals are transmitted to control system that is programmed to determine if the signals are indicative of an epileptic seizure. The system (1) controls the stimulation probe to cause the stimulation probe to apply stimulation to the brain, if (a) the sensed motion exceeds the predetermined threshold of movement characteristic and epileptic seizure and (b) the electrical activity of the brain is indicative of an epileptic seizure. Alternatively, the control system (2) controls the stimulation probe to cause the stimulation probe to avoid stimulation to the brain, if (a) the sensed motion exceeds the predetermined threshold of movement characteristic and epileptic seizure but (b) the electrical activity of the brain is not indicative of an epileptic seizure.

For a system intended to treat epilepsy, the system may also include a second stimulation probe configured for insertion into the patient's brain at a second location subject to stimulation affecting symptoms of an epileptic seizure, and the second stimulation probe may be operable to apply stimulation to a second portion of the patient's brain associated with an epileptic seizure. The control system is operable to control the second stimulation probe to cause the second stimulation probe to apply stimulation to a second portion of the patient's brain associated with an epileptic seizure. The control system is also further programmed to receive, from the motion sensor, signals corresponding to sensed movement of said body part after causing the first stimulation probe to apply stimulation to the first portion of the patient's brain associated with an epileptic seizure, and to receive, from the motion sensor, signals corresponding to sensed movement of said body part after causing the second stimulation probe to apply stimulation to the second portion of the patient's brain associated an epileptic seizure. The control system is also programmed to use this input to determine which application of stimulation is most effective at reducing aberrant movement associated with (characteristic of) an epileptic seizure, and thereafter continue causing the corresponding first or second stimulation probe to apply stimulation to the corresponding portion of the brain.

The system can include multiple sensor and stimulation probes and the control system can be further programmed to receive, signals corresponding to sensed movement of a body part after causing a first stimulation probe to apply stimulation to a first portion of the patient's brain associated with the movement disorder. The control system then receives signals corresponding to sensed movement of the body part after causing a second stimulation probe to apply stimulation to the second portion of the patient's brain associated with the movement disorder. The control system determine which stimulation is most effective at reducing aberrant movement associated with (characteristic of) the movement disorder, and thereafter continue causing the corresponding first or second stimulation probe to apply stimulation to the corresponding portion of the brain.

Operable coupling between the probes and the control system does not require a physical electrical connection through leads connected by wires to a pulse generator. Instead, the operable coupling of the probes to the control system is preferably achieved via wireless coupling. The lack of wires on the probe ends allows doctors to distribute more probes in order to allow for the establishment of a wireless network. The operable coupling is established via the network of probes implanted within the brain and a means of a remote wireless programming telecommunication scheme associated with the control system. The plurality of probes functions as “spokes” for wireless connection to the control system that functions as a “hubs” to create a network. The probes can communicate directly with the control system or alternatively directly with each other. Though wireless communication between the probes and the control system is preferred, the benefits of the system and mode of operation may be achieved with wireless or hard-wired connection between the probes and the control system.

In use, a surgeon will implant the sensor probes and stimulation probes within the patient's brain. The sensor probe receives and detects a condition of a patient that is then processed into an electrical signal corresponding to the brain signals from the probes and transmitted to the processor of the control system. The electronic signal is then subjected to an algorithm running on the control system that can detect patterns that result in the sensed electronic signals. When a pattern is detected, the control system sends a delivery or control signal via the transmitter to the stimulation probe which generates and delivers a predetermined stimulus (voltage, light vibration or current) to the patient's brain.

Preferably, the probes are implanted entirely within the brain and positioned in multiple regions of the brain subject to stimulation to affect symptoms of a disease such as Parkinson's disease, epilepsy, essential tremor and dystonia. Probes can also be positioned superficial to the scalp, between the dura and the cortex, or on the cortex. The senor probes are operated to general signals corresponding to the sensed biological signals of the patient's brain. The measured signal is transmitted to the control system. The processor of the control system interprets whether the measured signal is within a predetermined band of signal readings, or above or below a predetermined threshold for the signal readings. The control system is also operable to generate and transmit control signals to the probes, to cause the probes to transmit stimulation pulses to structures within the brain if the signal readings are determined to be outside of a predetermined range to effect therapeutic changes in native biological brain signals. The control system is programmed and operable to cause the probes to deliver a prescribed dosage of stimulation impulses to treat a variety of conditions and diseases such as Parkinson's disease, epilepsy, essential tremor and dystonia.

If the system is implemented in an embodiment which is configured to use motion signals obtained from motion sensors disposed on the body of the patient, the surgeon mounts an accelerometer on the patient's body at a location, which is known to exhibit distinct motions characteristic of the motion disorder. For example, the motion sensor can be secured to the wrist of the patient to sense tremors or other symptoms associated with movement disorders. These symptoms can be measured and transmitted as motion or acceleration signals to the control system in order for the control system to calculate the delivered electrical stimulation. The control system, in this embodiment, is programmed to receive and analyze the motion signals from the motion sensors to determine if the signals correspond to motion indicative of the motion disorder, and, based on this determination, generate and transmit control signals to the probes within the brain to cause the probes within the brain to deliver stimulation to the brain. Again, if the motion falls below a predetermined threshold, exceeds a predetermined threshold, or outside a predetermined range, as determined by the control system, the control system will generate and transmit control signals to the probe within the brain, to cause the probe to deliver more electrical stimulation, less stimulation, or stimulation of differing characteristics (frequency, pulse width, pulse frequency, or other pulse waveform parameters) to the brain. Additionally, the native biological brain signals and external probe measured signals can be shared either via an uplink to a network for sharing or analysis through machine learning and then modified by a down load back to the user.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A system for treating movement disorders with stimulation of a patient's brain, said system comprising:
a stimulation probe configured for insertion into the patient's brain at a location subject to stimulation affecting symptoms of the movement disorder, said stimulation probe operable to apply stimulation to a portion of the patient's brain associated with the movement disorder;
a motion sensor configured for placement on a body part of the patient, said body part subject to aberrant movement associated with (characteristic of) the movement disorder, said motion sensor operable to sense movement of said body part, generate signals corresponding to sensed movement of said body part, and transmit said signals corresponding to sensed movement of said body part;
a control system operable to receive signals corresponding to the sensed movement of the body part, and operable to control the stimulation probe to cause the stimulation probe to apply stimulation to a portion of the patient's brain associated with the movement disorder, said control system programmed to
(1) receive signals corresponding to sensed movement of said body part and determine if the signals corresponding to sensed movement of said body part are indicative of aberrant movement associated with (characteristic of) the movement disorder, and, if the signals corresponding to sensed movement of said body part are indicative of aberrant movement associated with (characteristic of) the movement disorder,
(2) thereafter control the stimulation probe to cause the stimulation probe to apply stimulation to a portion of the patient's brain associated with the movement disorder.

2. The system of claim 1, wherein:
the control system is further programmed to (3) after causing the stimulation probe to apply stimulation, continue to receive signals corresponding to sensed movement of said body part and conditionally (4) determine that the signals corresponding to sensed movement of said body part are indicative of aberrant movement associated with (characteristic of) the movement disorder, and further causing the stimulation probe to cause the stimulation probe apply stimulation or (5) determine that the signals corresponding to sensed movement of said body part are NOT indicative of aberrant movement associated with (characteristic of) the movement disorder, and cease causing the stimulation probe to cause the stimulation probe apply stimulation.

3. The system of claim 1 or 2, wherein: a motion sensor configured for placement on the wrist or hand of a patient, and the control system is programmed to determine if the sensed motion exceeds a predetermined threshold of tremor characteristic of tremor due to Parkinson's disease and cause the stimulation probe to apply stimulation to the brain until the sensed motion falls below the predetermined threshold.

4. The system of claim 1 or 2, wherein a motion sensor configured for placement on the wrist or hand of a patient, and the control system is programmed to determine if the sensed motion exceeds a predetermined threshold of tremor characteristic and is of tremor due to essential tremor and cause the stimulation probe to apply stimulation to the brain until the sensed motion falls below the predetermined threshold.

5. The system of claim 1 or 2, wherein a motion sensor configured for placement on an extremity of a patient, and the control system is programmed to determine if the sensed motion exceeds a predetermined threshold of movement characteristic and epileptic seizure and cause the stimulation probe to apply stimulation to the brain until the sensed motion falls below the predetermined threshold.

6. The system of claim 5, further comprising:
a sensor probe configured for insertion into the patient's brain, and operable to detect electrical activity of the brain indicative of an epileptic seizure, generate signals corresponding to electrical activity of the brain, and transmit said signals corresponding to electrical activity of the brain, and
the control system is further operable to receive said signals corresponding to electrical activity of the brain, and further programmed to determine if said signals corresponding to electrical activity of the brain are indicative of an epileptic seizure, and, conditionally
(1) control the stimulation probe to cause the stimulation probe to apply stimulation to the brain, if (a) the sensed motion exceeds the predetermined threshold of movement characteristic and epileptic seizure and (b) the electrical activity of the brain is indicative of an epileptic seizure or
(2) control the stimulation probe to cause the stimulation probe to avoid stimulation to the brain, if (a) the sensed motion exceeds the predetermined threshold of movement characteristic and epileptic seizure but (b) the electrical activity of the brain is not indicative of an epileptic seizure.

7. The system of claim 1 further comprising:
a second stimulation probe configured for insertion into the patient's brain at a second location subject to stimulation affecting symptoms of the movement disorder, said second stimulation probe operable to apply stimulation to a second portion of the patient's brain associated with the movement disorder; wherein
the control system is further operable to control the second stimulation probe to cause the second stimulation probe to apply stimulation to a second portion of the patient's brain associated with the movement disorder, and
the control system further programmed to receive, from the motion sensor, signals corresponding to sensed movement of said body part after causing the first stimulation probe to apply stimulation to the first portion of the patient's brain associated with the movement disorder, and receive, from the motion sensor, signals corresponding to sensed movement of said body part after causing the second stimulation probe to apply stimulation to the second portion of the patient's brain associated with the movement disorder, and further programmed to determine which application of stimulation is most effective at reducing aberrant movement associated with (characteristic of) the movement disorder, and thereafter continue causing the corresponding first or second stimulation probe to apply stimulation to the corresponding portion of the brain.

8. The system of claim 6 further comprising:
a second stimulation probe configured for insertion into the patient's brain at a second location subject to stimulation affecting symptoms of an epileptic seizure, said second stimulation probe operable to apply stimulation to a second portion of the patient's brain associated with an epileptic seizure; wherein
the control system is further operable to control the second stimulation probe to cause the second stimulation probe to apply stimulation to a second portion of the patient's brain associated with an epileptic seizure, and
the control system further is programmed to receive, from the motion sensor, signals corresponding to sensed movement of said body part after causing the first stimulation probe to apply stimulation to the first portion of the patient's brain associated with an epileptic seizure, and receive, from the motion sensor, signals corresponding to sensed movement of said body part after causing the second stimulation probe to apply stimulation to the second portion of the patient's brain associated an epileptic seizure, and further programmed to determine which application of stimulation is most effective at reducing aberrant movement associated with (characteristic of) an epileptic seizure, and thereafter continue causing the corresponding first or second stimulation probe to apply stimulation to the corresponding portion of the brain.

9. A method treating movement disorders with stimulation of a patient's brain, said method comprising:
providing a plurality of sensor probes adapted to sense brain signals and generate electronic signals corresponding to brain signals;
providing a plurality of stimulation probes adapted to deliver electrical stimulation to the patient's brain;
inserting the sensor probes and the stimulation probes within into the patient's brain in multiple locations of the patient's brain;
transmitting the generated electronic signals from the sensor probe to the control system to determine if the signals are within a predetermined threshold; and
sending a control signal from the control system to the stimulation probe to apply stimulation to the patient's brain in response to the generated electronic signals.
